(19) United States
(12) Patent Application Publication
(10) Pub. No.: US 2017/0347929 A1
(43) Pub. Date: Dec. 7, 2017

(54) ELECTRODE ASSEMBLY

(71) Applicant: Nanoflex Limited, Daresbury, Cheshire (GB)

(72) Inventor: Neville John FREEMAN, Tarvin, Chester (GB)

(73) Assignee: Nanoflex Limited, Daresbury, Cheshire (GB)

(21) Appl. No.: 15/527,761

(22) PCT Filed: Nov. 18, 2015
(86) PCT No.: PCT/GB2015/053499

§ 371 Date: May 18, 2017

(30) Foreign Application Priority Data
Nov. 18, 2014 (GB) 1420477.0

(51) Int. Cl. A61B 5/145; A61B 5/1486; A61B 5/00

(52) U.S. Cl. CPC A61B 5/14532; A61B 5/4839; A61B 5/14865; A61B 5/1486; A61B 2562/0285; A61B 2562/046

(57) ABSTRACT

The present invention relates to an electrode assembly (eg a nanoelectrode assembly), to an electrochemical glucose biosensor comprising the electrode assembly and to an apparatus for combating (eg management of) diabetes mellitus which comprises the electrochemical glucose biosensor.

The present invention relates to an electrode assembly (eg a nanoelectrode assembly), to an electrochemical glucose biosensor comprising the electrode assembly and to an apparatus for combating (eg management of) diabetes mellitus which comprises the electrochemical glucose biosensor.

Conventional glucose biosensors using glucose oxidase saturate at a level of around 2 mM normally due to their response being limited by low levels of physiological oxygen (the so-called “oxygen deficit”). This level of glucose is dangerously low in a diabetic. An approach to improving the oxygen deficit is to add a glucose membrane which selectively restricts the amount of glucose relative to oxygen reaching the electrode (ie increases the oxygen/glucose permeability ratio). This has the effect of making the sensor respond to higher levels of glucose but significantly reduces the sensitivity and speed of response which makes it difficult to measure glucose below a level of 4 mM. An alternative approach to improving the oxygen deficit is to deploy a mediator which takes over the role of oxygen by carrying electrons from the active site of glucose oxidase to the electrode surface. Known mediators include transition metal complexes which are poorly tolerable physiologically and often toxic. Thus the identification of an improved approach to lowering measurement thresholds is critical to the rapidly growing area of continuous glucose monitoring (CGM).

The present invention is based on the recognition that a certain electrode assembly (eg nanoelectrode assembly) upon which is immobilized glucose oxidase may be able to perform without a glucose-restricting membrane over a significantly wider dynamic range of glucose levels than would have been expected on the basis of physiological constraints. In particular, it has been determined that the electrode assembly can perform over the entire operational range of glucose oxidase (0 to 30 mM) in (for example) normally aspirated aqueous solutions and with a limit of detection as low as 0.5 μM.

Thus viewed from a first aspect the present invention provides a nanoelectrode assembly having a laminate structure comprising:
a first insulating capping layer;
a first conducting layer capped by the first insulating capping layer and substantially sandwiched or encapsulated by at least the first insulating capping layer such as to leave exposed only an electrical contact surface; and
an array of etched voids extending through at least the first insulating capping layer and the first conducting layer, wherein each void is partly bound by a surface of the first conducting layer which acts as an internal submicron electrode upon or adjacent to which is immobilised a glucose sensitive enzyme, wherein in use a glucose-containing bodily fluid passes into the etched voids for exposure to the immobilised glucose sensitive enzyme.

In a preferred embodiment of the nanoelectrode assembly in use, relative mass transfer of glucose and oxygen from the bodily fluid to the immobilised glucose sensitive enzyme is unselective.

In a preferred embodiment of the nanoelectrode assembly in use, relative mass transfer of glucose and oxygen from the bodily fluid to the immobilised glucose sensitive enzyme is interventionless.

In a preferred embodiment of the nanoelectrode assembly in use, relative mass transfer of glucose and oxygen from the bodily fluid to the immobilised glucose sensitive enzyme is unimpeded.

Typically the nanoelectrode assembly is free of any system which is glucose flux-limiting or glucose diffusion-controlling (eg a glucose membrane).

Preferably the nanoelectrode assembly is free of a glucose-restricting membrane (eg is membraneless).

The absence of intervention, impediment or selectivity over relative mass transfer of glucose and oxygen (eg by a membrane) leads advantageously to more rapid equilibration with the bodily fluid (eg interstitial fluid) and a faster response.

In a preferred embodiment of the nanoelectrode assembly, the immobilised glucose sensitive enzyme is oxygen-mediated (eg substantially solely oxygen-mediated). This embodiment advantageously improves physiological tolerability and alleviates safety concerns.

The nanoelectrode assembly may be free of an exogenous mediator such as a synthetic mediator (eg an inorganic mediator such as a transition metal complex).

Alternatively the glucose sensitive enzyme may be co-immobilised with a mediator such as a synthetic mediator (eg an inorganic mediator such as a transition metal complex).

In a preferred embodiment, the glucose sensitive enzyme is glucose oxidase.

The electrical contact surface may be part of the first conducting layer or may be connected to the first conducting layer. The electrical contact surface may be a peripheral contact edge such as a square contact edge of the conducting layer. The electrical contact surface may be a wide area electrical contact surface (eg the electrical contact surface may extend along substantially the entire length of the periphery of the nanoelectrode assembly). The electrical contact surface may be substantially T-shaped. The electrical contact surface may be an electrical contact lip. The electrical contact surface allows simple and reliable connection of each internal submicron electrode to external instrumentation eg external circuitry such as a potentiostat, handheld meter or monitoring device for example.

Typically the nanoelectrode assembly has at least one dimension (eg one or two dimensions) on the nanometer scale. This dimension is often referred to as the critical dimension and largely controls the electrochemical response. The critical dimension may be 100 nm or less.

The layers of the laminate structure may be successively fabricated (eg cast, spun, sputtered, grown or deposited) layer-by-layer according to standard techniques.

Preferably the nanoelectrode assembly comprises: a plurality of conducting layers (which may be the same or different) including the first conducting layer and a plurality of insulating capping layers (which may be the same or different) including the first insulating capping layer, wherein the plurality of conducting layers and the plurality of insulating capping layers are alternating in the laminate structure, wherein each conducting layer is sandwiched or encapsulated to leave exposed only an electrical contact surface and the array of etched voids extends through the plurality of insulating capping layers and the plurality of conducting layers, wherein each void is partly bound by a surface of each of the plurality of conducting layers which acts as an internal submicron electrode upon or adjacent to which is immobilised the glucose sensitive enzyme.

The number of internal submicron electrodes in each void may be three, four or five (or more). Such embodiments may be formed by successive lamination (eg deposition or growth) of the conducting layers and insulating capping layers. The dimensions and absolute spatial locations within the void and relative spatial locations of each of the internal submicron electrodes may be precisely defined.

Preferably the nanoelectrode assembly further comprises: a second conducting layer, wherein the first conducting layer is sandwiched or encapsulated to leave exposed only a first electrical contact surface and the second conducting layer is sandwiched or encapsulated to leave exposed only a second electrical contact surface, wherein the array of etched voids extends through the first conducting layer, the first insulating capping layer and the second conducting layer, wherein each void is partly bound by a surface of the first conducting layer which acts as an internal submicron electrode upon or adjacent to which is immobilised the glucose sensitive enzyme and/or by a surface of the second conducting layer which acts as an internal submicron electrode upon or adjacent to which is immobilised the glucose sensitive enzyme. The first conducting layer and second conducting layer may be substantially coplanar (eg laterally spaced apart). The first conducting layer and second conducting layer may be non-coplanar (eg axially spaced apart (preferably substantially co-axially spaced apart) or radially spaced apart (preferably concentrically radially spaced apart)). This may require multilevel metal interconnect.

Preferably the nanoelectrode assembly comprises: a second conducting layer and a second insulating capping layer, wherein the first conducting layer is sandwiched or encapsulated to leave exposed only a first electrical contact surface and the second conducting layer is sandwiched or encapsulated to leave exposed only a second electrical contact surface, wherein the array of etched voids extends through the first conducting layer, the first insulating capping layer, the second conducting layer and the second insulating capping layer, wherein each void is partly bound by a surface of the first conducting layer which acts as an internal submicron electrode upon or adjacent to which is immobilised the glucose sensitive enzyme and/or by a surface of the second conducting layer which acts as an internal submicron electrode upon or adjacent to which is immobilised the glucose sensitive enzyme. The first conducting layer and second conducting layer may be substantially coplanar (eg laterally spaced apart). The first conducting layer and second conducting layer may be non-coplanar (eg axially spaced apart (preferably substantially co-axially spaced apart) or radially spaced apart (preferably concentrically radially spaced apart)). This may require multilevel metal interconnect.

Preferably the array of etched voids is a plurality of discrete sub-arrays of etched voids. The array (or each sub-array) may be a linear or staggered (eg herringbone) pattern. The array (or each sub-array) may be a cubic pattern. The array (or each sub-array) may be a multi-dimensional (eg bi-dimensional) array.

The array of voids may be mechanically or chemically etched. Each void may be an aperture, through-hole, well, tube, capillary, pore, bore or trough. Preferably each etched void is a well. The well may terminate in an insulating capping layer or insulating substrate layer. The well may terminate in a conducting layer which provides an internal submicron electrode in the base of the well.

The lateral dimension (d_{w}) and shape of a void determines the distance between opposite faces of the internal submicron electrode.

The cross-sectional shape of the void may be regular. For example, the cross-sectional shape of the void may be substantially circular and the lateral dimension is the diameter. For example, the cross-sectional shape of the void may be substantially square and the lateral dimension is the width.

The lateral dimension d_{w} (eg width or diameter) of each void (which may be the same or different) is typically 100 nm or more.

The depth of the void is the etch depth (d_{d}). The position of the n^{th} internal submicron electrode at a specified depth (dₙ) in the void (ie the distance from the aperture opening to the closest edge of the n^{th} electrode) is determined by the width of the insulating capping layer(s). The thickness of the internal submicron electrode (wₙ) and its position within the void (defined by dₙ, d_{d} and wₙ) can be independently controlled on the nanoscale.

The etch depth (d_{d}) of each void (which may be the same or different) is typically 10000 microns or less, preferably 0.0003 to 1000 microns, particularly preferably 0.05 to 100 microns, more preferably 0.01 to 10 micron.

The plurality of voids can be arranged in an array with a precisely defined separation or pitch (x and y which may be the same or different). The pitch is typically 100 nm or more.

The (or each) conducting layer may be a substantially planar or cylindrical conducting layer.

Preferably the (or each) conducting layer is a substantially planar conducting layer.

The (or each) conducting layer may be substantially T-shaped, serpentine or digitated.

The (or each) conducting layer may be metallic. The conducting layer may be composed of a noble metal such as gold or silver or a metal nitride (eg titanium nitride). The (or each) conducting layer may be functionalised (eg chemically or biologically functionalised).

The (or each) conducting layer may be a composite (eg a composite of a nanoparticle, nanowire or nanoconnector). For example, the (or each) conducting layer may comprise (or consist of) carbon nanotubes or metal (eg gold) nanoparticles.

The thickness (wₙ) of the n^{th} conducting layer may be determined by fabrication at atomic scale resolution (where atomic scale means a thickness of at least an atom or more). The thickness (wₙ) of the (or each) conducting layer (which may be the same or different) may be 0.10 nm or more, preferably in the range 0.10 to 990 nm, particularly preferably 0.10 to 500 nm, more preferably 0.10 to 250 nm, even more preferably 0.10 to 100 nm.

The (or each) insulating capping layer may be polymeric. The thickness of the (or each) insulating capping layer (which may be the same or different) may be 0.10 nm or more, preferably in the range 0.10 to 5000 nm, particularly preferably 0.10 to 2000 nm, more preferably 0.10 to 990 nm, most preferably 0.10 to 500 nm.

The depth of the first internal submicron electrode (ie the internal submicron electrode closest to the hole edge) (d₁) is typically 1000 microns or less, preferably 0.0001 to 100 microns, particularly preferably 0.0001 to 10 microns, more preferably 0.0001 to 1 micron, most preferably 0.0001 to 0.5 microns.

The (or each) internal submicron electrode is typically partly or wholly annular.

In a first preferred embodiment, the first conducting layer is substantially sandwiched or encapsulated by only the first insulating capping layer such as to leave exposed only an electrical contact surface of the first conducting layer, wherein the array of etched voids extends through only the first insulating capping layer and the first conducting layer.

In a second preferred embodiment, the electrode further comprises:
an insulating substrate layer, wherein the first conducting layer is fabricated on the insulating substrate layer and is substantially sandwiched or encapsulated by the first insulating capping layer and the insulating substrate layer such as to leave exposed only an electrical contact surface of the first conducting layer.

In a third preferred embodiment, the electrode further comprises:
an insulating substrate layer;
a second insulating capping layer fabricated on the insulating substrate layer,

wherein the first conducting layer is fabricated on the second insulating capping layer and is substantially sandwiched or encapsulated by the first insulating capping layer and the second insulating capping layer such as to leave exposed only an electrical contact surface of the first conducting layer.

In a fourth preferred embodiment, the electrode further comprises:
an insulating substrate layer;
a second insulating capping layer,

wherein the first conducting layer is fabricated on the second insulating capping layer and is substantially sandwiched or encapsulated by the first insulating capping layer and the second insulating capping layer such as to leave exposed only an electrical contact surface of the first conducting layer;
a second conducting layer,

wherein the second conducting layer is fabricated on the insulating substrate layer and is substantially sandwiched or encapsulated by the second insulating capping layer and the insulating substrate layer such as to leave exposed only an electrical contact surface of the second conducting layer,

wherein the array of etched voids extends through at least the first insulating capping layer, the first conducting layer and the second insulating capping layer, wherein each void is partly bound by a surface of the first conducting layer which acts as an internal submicron electrode upon or adjacent to which is immobilised the glucose sensitive enzyme.

Particularly preferably the array of etched voids extends through only the first insulating capping layer, the first conducting layer and the second insulating capping layer.

Particularly preferably the array of etched voids extends through the first insulating capping layer, the first conducting layer, the second insulating capping layer and the second conducting layer, wherein each void is partly bound by a surface of the first conducting layer which acts as a first internal submicron electrode upon or adjacent to which is immobilised the glucose sensitive enzyme and by a surface of the second conducting layer which acts as a second internal submicron electrode optionally (but preferably) upon or adjacent to which is immobilised the glucose sensitive enzyme.

In a fifth preferred embodiment, the electrode further comprises:
an insulating substrate layer;
a second conducting layer,

wherein the first conducting layer is digitated and the second conducting layer is digitated, wherein the first conducting layer and the second conducting layer are interdigitally fabricated on the insulating substrate layer and are substantially sandwiched or encapsulated by the first insulating capping layer and the insulating substrate layer such as to leave exposed only an electrical contact surface of the first conducting layer and an electrical contact surface of the second conducting layer,

wherein the array of etched voids extends through the first insulating capping layer, the first conducting layer and the second conducting layer, wherein each void is partly bound by a surface of the first conducting layer which acts as a first internal submicron electrode upon or adjacent to which is immobilised the glucose sensitive enzyme and is partly bound by a surface of the second conducting layer which acts as a second internal submicron electrode optionally (but preferably) upon or adjacent to which is immobilised the glucose sensitive enzyme.

In a sixth preferred embodiment, the electrode further comprises:
an insulating substrate layer;
a second conducting layer,

wherein the second conducting layer is substantially coplanar with the first conducting layer, wherein each of the first conducting layer and the second conducting layer is capped by the first insulating capping layer and is substantially sandwiched or encapsulated by at least the first insulating capping layer such as to leave exposed only an electrical contact surface of the first conducting layer and an electrical contact surface of the second conducting layer respectively,

wherein one or more first etched voids extend through the first insulating capping layer and the first conducting layer and one or more second etched voids extend through the first insulating capping layer and the second conducting layer, wherein each first etched void is partly bound by a surface of the first conducting layer which acts as an internal submicron electrode upon or adjacent to which is immobilised the glucose sensitive enzyme and each second etched void is partly bound by a surface of the second conducting layer which acts as an internal submicron electrode upon or adjacent to which is immobilised the glucose sensitive enzyme.

Particularly preferably each of the first conducting layer and the second conducting layer is substantially sandwiched or encapsulated by only the first insulating capping layer such as to leave exposed only an electrical contact surface of the first conducting layer and an electrical contact surface of the second conducting layer respectively.

The insulating substrate layer is typically composed of silicon, silicon dioxide, silicon nitride or a polymeric material.

The laminate structure may be substantially planar, cylindrical, box cross-section, hemispherical or spherical. A cylindrical, hemispherical or spherical laminate structure may have a hollow or solid core.

For example, the laminate structure may be a fibre which may have a hollow or solid core with a diameter of 1 micron or more.

For example, the laminate structure may be a slide, taper, plate or tape which may have a width of 1 micron or more.

The nanoelectrode assembly may be equipped with a permselective membrane.

The bodily fluid may be blood, urine, intraocular fluid (eg aqueous humour), lachrymal fluid, saliva, sweat or interstitial fluid.

Viewed from a further aspect the present invention provides an electrochemical glucose biosensor comprising:
a nanoelectrode assembly as hereinbefore defined operable as a working electrode; and
a reference electrode and a counter electrode or a combined counter reference electrode.

The electrochemical glucose biosensor is typically amperometric.

The electrochemical glucose biosensor may be topically mountable (eg skin-mountable).

The electrochemical glucose biosensor may be implantable or injectable into a body of a subject. The electrochemical glucose biosensor may be implantable intravenously or subcutaneously. Preferably the electrochemical glucose biosensor is implantable subcutaneously.

The electrochemical glucose biosensor may be predominantly needle-like.

Viewed from a yet further aspect the present invention provides the use of an electrochemical glucose biosensor as hereinbefore defined for continuously measuring the glucose level in a subject.

Viewed from a still yet further aspect the present invention provides an apparatus for combating (eg treating or preventing) diabetes mellitus in a subject comprising:
an electrochemical glucose biosensor as hereinbefore defined for continuously measuring the glucose level in the subject;
a signal generating device for generating an actuating signal in response to the glucose level exceeding a threshold; and
a delivery device for delivering insulin to the subject in response to the actuating signal, wherein in use the electrochemical glucose biosensor, signal generating device and delivery device communicate in a closed loop.

The delivery device is typically an insulin pump.

Preferably the electrochemical glucose biosensor is subcutaneously implanted or topically mounted.

The present invention will now be described in a non-limitative sense with reference to the accompanying Examples and Figures in which:

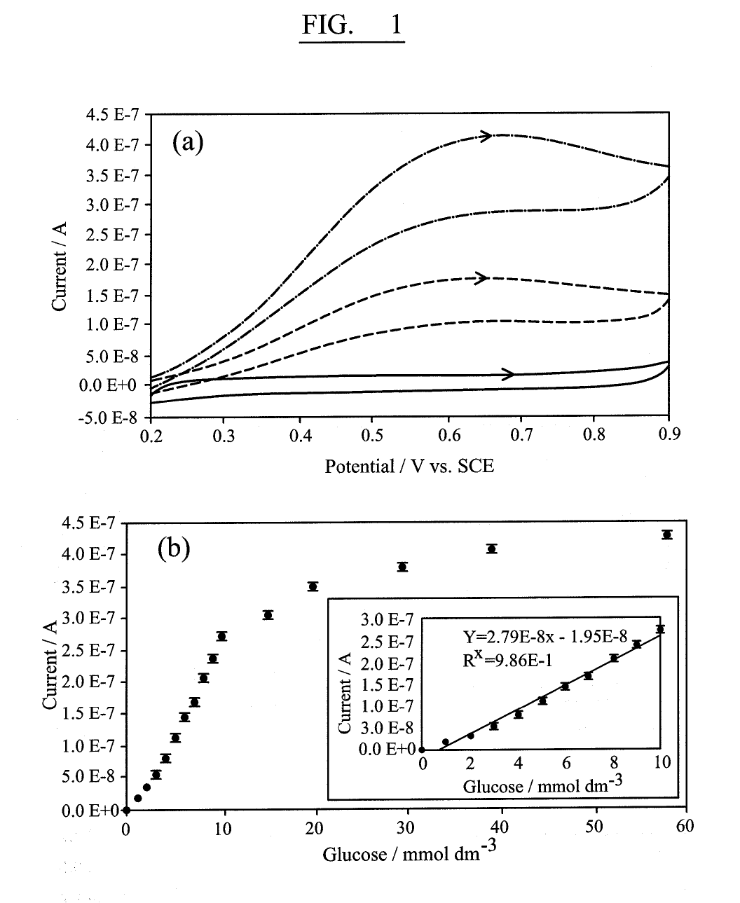

FIG. 1: An illustration of the typical response of an embodiment of the electrochemical glucose biosensor of the invention;

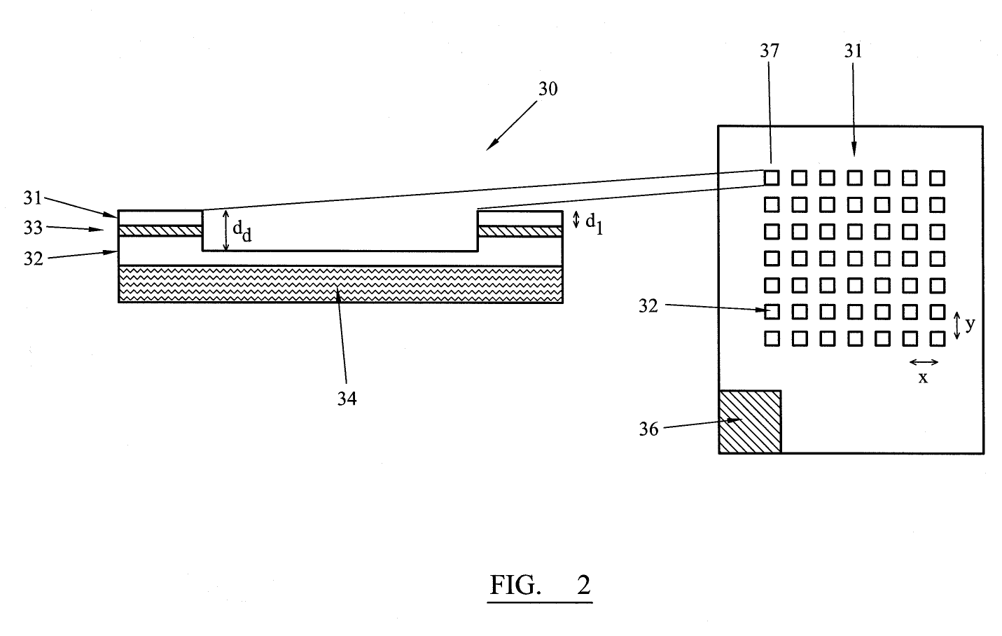

FIG. 2: A schematic partial cross-section and top view of a first embodiment of the nanoelectrode assembly of the invention;

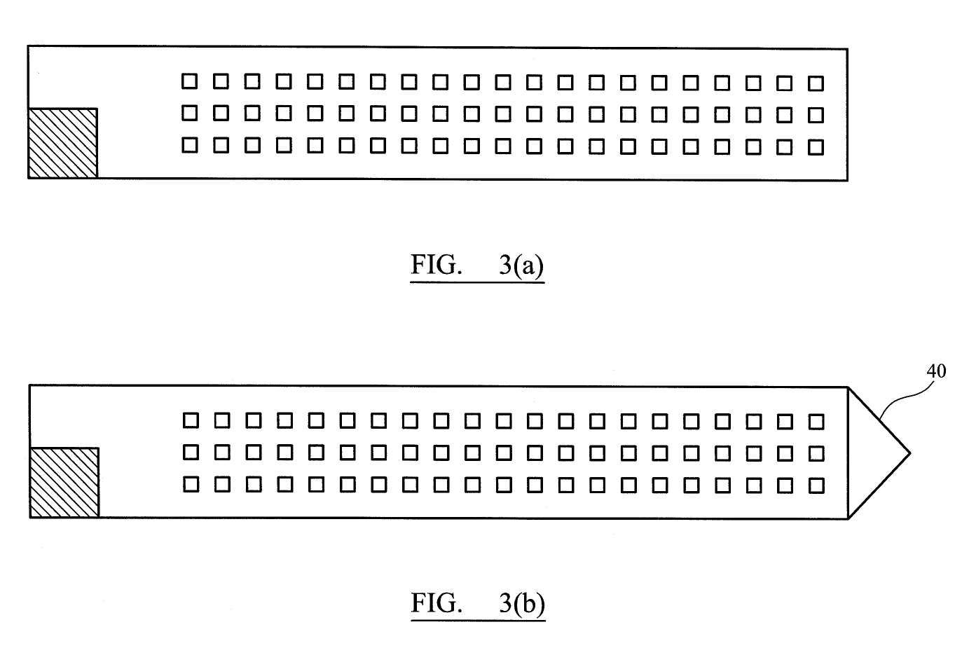

FIGS. 3a-b: A top view of two variations of the nanoelectrode assembly of the first embodiment;

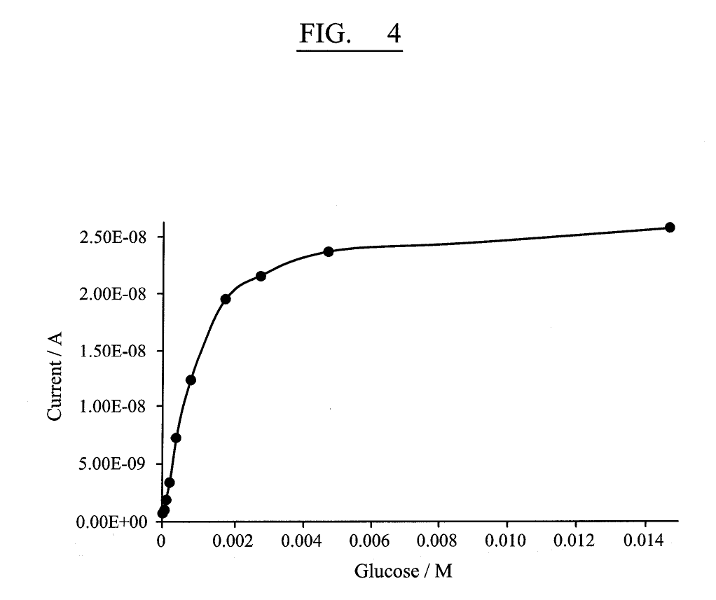

FIG. 4: A response from a 150 μm needle-like sensor;

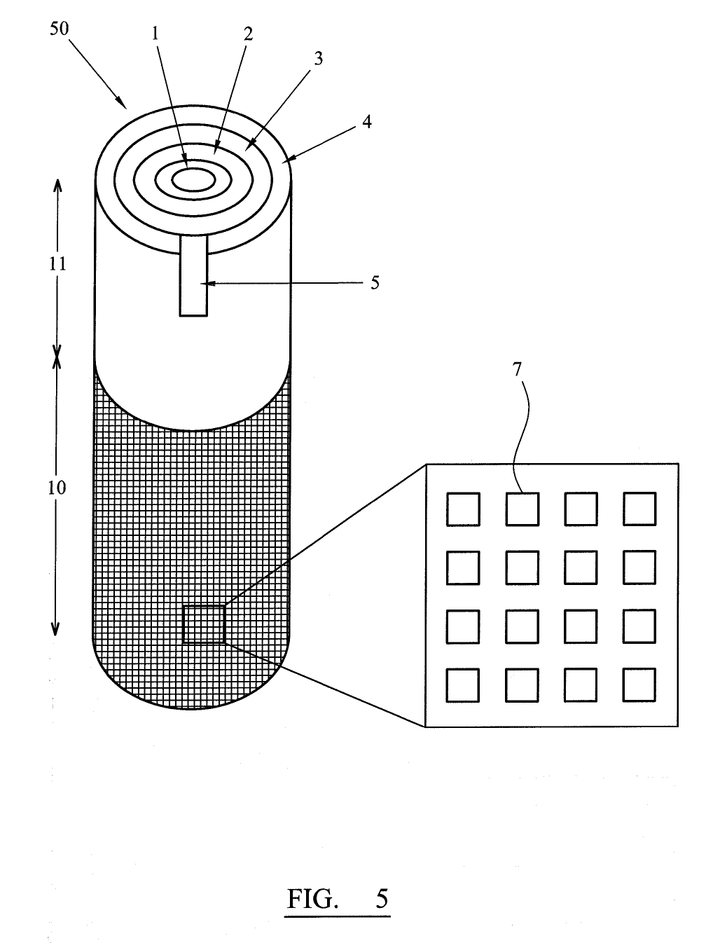

FIG. 5: A schematic perspective view of a second embodiment of the nanoelectrode assembly of the invention; and

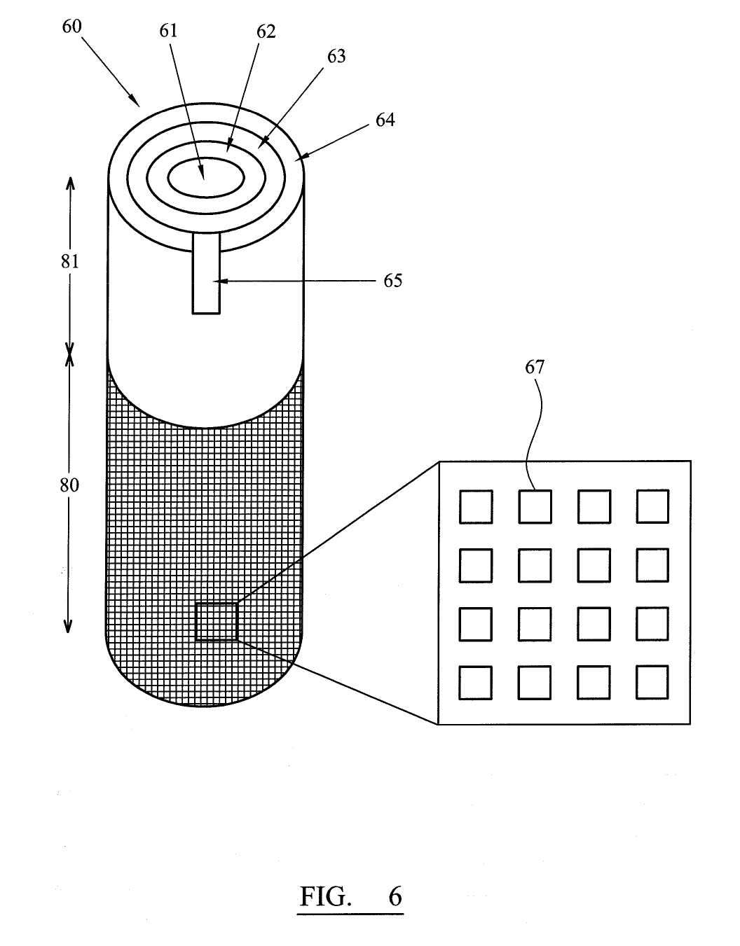

FIG. 6: A schematic perspective view of a third embodiment of the nanoelectrode assembly of the invention.

### EXAMPLE 1

A commercial electrode (303D platinum 50 nm nanoband electrode, NanoFlex Ltd (UK)) was used to prepare a glucose oxidase (GOx)-immobilised working electrode mediated by oxygen (as described below) for use in a three electrode electrochemical cell with a saturated calomel electrode (Scientific Laboratory Supplies (UK)) and a 0.5 mm diameter platinum wire counter electrode (Fisher Scientific (UK)).

### Conditioning of the Electrode

The commercial electrode was cleaned by soaking in acetone for 10 minutes, iso-propanol for 10 minutes and 18.2 MΩ deionised water for 10 minutes and then dried under nitrogen.

The electrode was conditioned electrochemically using cyclic voltammetry. Firstly 50 cm³ of 0.1 mol dm⁻³ citrate buffer was placed in an electrochemical cell and appropriate connections were made with the potentiostat. The electrode was conditioned using parameters detailed in Table 1.

TABLE 1
Conditioning parameters for citrate buffer

| Conditioning Solution | 0.1 mol dm⁻³ citrate buffer (50 cm³) |
|---|---|
| Start E (V) | 0.5 |
| High E (V) | 1.7 |
| Low E (V) | −1.2 |
| Scans | 10 |
| Scan Rate (V s⁻¹) | 0.1 |
| Initial Polarity | Positive |
| Step (V) | 0.001 |

The electrode was then removed from the electrochemical cell and rinsed with copious amounts of 18.2 MΩ deionised water. The electrode was then immersed in 50 cm³ of 0.05 mol dm⁻³ sulfuric acid solution and conditioned using parameters detailed in Table 2.

TABLE 2
Conditioning parameters for sulfuric acid

| Conditioning Solution | 0.05 mol dm⁻³ sulfuric acid (50 cm³) |
|---|---|
| Start E (V) | 0.5 |
| High E (V) | 1.0 |
| Low E (V) | −1.2 |
| Scans | 10 |
| Scan Rate (V s⁻¹) | 0.1 |
| Initial Polarity | Positive |
| Step (V) | 0.001 |

Preparation of the GOx-Immobilised Working Electrode

The conditioned electrode was placed in a separate glass beaker with the array facing upwards. 2 cm³ of concentrated sulfuric acid (99.99% purity) was pipetted onto the electrode to cover the entire surface and left for 5 minutes to remove all traces of organics. The electrode was then rinsed in copious amounts of 18.2 MΩ deionised water and dried under nitrogen.

The electrode was immersed in 50 μmol dm⁻³ ethanolic mercaptohexyl amine (MHA) prepared in a glass container. The container was back-filled with dry nitrogen and the cap was sealed and wrapped with parafilm. The electrode was stored in this condition at room temperature (21° C.) for 24 hours in the dark.

The thiolated electrode was then taken out of the ethanolic MHA solution and rinsed in ethanol for 10-15 seconds using a clean solvent bottle to remove excess thiol. It was then immediately rinsed in 18.2 MΩ deionised water and then dried under dry nitrogen.

150 μL of a 5% solution of glutaraldehyde prepared in deionised water was pipetted onto the electrode which was then left to incubate for 45 minutes at room temperature. 40 mg/mL GOx was then made up in 0.01 mol dm⁻³ phosphate buffered saline (PBS) and 150 uL of the GOx solution was added onto the electrode. The electrode was left to incubate for 2 hours at room temperature. The GOx solution was then removed and the electrode was rinsed with 0.01 mol dm⁻³ PBS (pH 7.0) and left immersed in 0.01 mol dm⁻³ PBS until used.

The working electrode is an example of a first embodiment of the nanoelectrode assembly of the invention with internal submicron electrodes upon each of which is immobilised GOx. The nanoelectrode assembly 30 is illustrated schematically in part cross-section and from the top in FIG. 2 and is a planar laminate structure which has a substantially square (plate-like) profile. The nanoelectrode assembly 30 comprises a conducting layer 33 of platinum (thickness w₁=50 nm) deposited on an insulating capping layer 32 of silicon oxide which is thermally grown on a silicon wafer substrate 34. An insulating capping layer 31 is deposited over the extent of the conducting layer 33 with the exception of one corner 36 which is left exposed to act as an electrical contact for direct and simple connection to an electrochemical measuring device (eg potentiostat). An array of square voids 37 is etched through insulating capping layer 31 and conducting layer 33 and partly through insulating capping layer 32 to an etch depth (d_{d}) which is short of the substrate 34.

Performance of the GOx Immobilised Working Electrode

Table 4 provides details of the reagents and the parameters used for glucose detection.

TABLE 4
Sample preparation
Blank electrolyte

| | |
|---|---|
| 250 cm³ of 1 × 10−4 mol | 1. Weigh out 0.345 g of sodium phosphate |
| dm⁻³ ferrocenemethanol | monobasic and 0.445 g of sodium phosphate |
| in 0.01 mol dm⁻³ | dibasic and transfer to a 250 cm³ volumetric |
| Sodium Phosphate Buffer | flask and make up to mark with deionised |
| Solution (PBS) (pH 7.0). | water. Mix thoroughly to dissolve (final |
| | concentration = 0.01 mol dm⁻³ PBS) |
| | 2. Weigh out 0.0054 g of ferrocenemethanol |
| | and transfer to a separate 250 cm³ volumetric |
| | flask and make up to mark with the 0.01 mol |
| | dm⁻³ PBS. Sonicate for 30 minutes to dissolve |
| | completely. |
| β-D-glucose stock | Weigh out 0.3603 g of D-glucose into a 20 cm³ |
| solution 1 | volumetric flask and make up to the |
| 20 cm³ of 0.1 mol | mark with 0.01 mol dm⁻³ PBS. Mutarotate |
| dm⁻³ β-D-glucose | the solution at 4° C. for 24 hours to gain |
| | equilibration between α- and β-forms of |
| | glucose. |
| β-D-glucose stock | Pipette 2 mL of the β-D-glucose stock |
| solution 2 | solution 1 into a 20 cm³ volumetric flask |
| 20 cm³ of 0.01 mol | and make up to the mark with 0.01 mol dm⁻³ |
| dm⁻³ β-D-glucose | PBS. Mutarotate the solution at 4° C. for 24 |
| | hours to gain equilibration between α- and |
| | β-forms of glucose. |

Parameters for Cyclic Voltammetry

| | |
|---|---|
| Start E (V) | 0.2 |
| High E (V) | 0.9 |
| Low E (V) | −0.2 |
| Scans | 1 |
| Scan Rate (V s⁻¹) | 0.05 |
| Initial Polarity | Positive |
| Step (V) | 0.001 |

Sensor Performance

The electrochemical cell was filled with 50 cm³ of the blank electrolyte solution. The solution was used as received (no aeration). A measurement was first taken in the blank using parameters detailed in Table 4. The solution was mixed thoroughly with a magnetic stirrer for 1 minute at 500 rpm and a measurement was taken using parameters in Table 4. Glucose was added sequentially up to a concentration of 60 mmol dm⁻³ and the current response was measured.

It can be seen from FIG. 1 that the electrode responded across the full concentration range over which the enzyme is capable of working. It is therefore apparent that it is possible to use the enzyme in conditions similar to the expected physiological concentrations of oxygen where glucose concentration remains the limiting factor and therefore enables the device to act as a glucose biosensor without the need for the use of glucose-restricting membranes. A limit of detection of 2.6 μmol dm⁻³ was calculated using the IUPAC methodology.

In alternative embodiments shown in FIGS. 3a-b, the profile of the planar laminate structure is substantially rectangular (strip-like) and may incorporate an end projection 40 to facilitate implantation (needle-like—see FIG. 3b). FIG. 4 illustrates the response from a 150 μm needle-like electrochemical glucose biosensor.

### EXAMPLE 2

FIG. 5 illustrates a schematic perspective view of a second embodiment of the nanoelectrode assembly of the invention which is a substantially cylindrical laminate structure. The nanoelectrode assembly 50 comprises a conducting layer 3 deposited on an insulating capping layer 2 which itself is on a hollow cylindrical support 1. An insulating capping layer 4 is deposited over the extent of the conducting layer 3 and incorporates an electrical contact 5 for direct and simple connection to an electrochemical measuring device (eg potentiostat). An array of square voids 7 is etched through insulating capping layer 4 and conducting layer 3 and partly through insulating capping layer 2 to an etch depth which is short of the hollow cylindrical support 1. The array of square voids 7 extends over only a lower portion 10 of the cylindrical laminate structure. The lower portion 10 is selectively implantable into the body to leave exposed an upper portion 11. The hollow cylindrical support 1 defines a receiving bore for gas or fluid delivery.

### EXAMPLE 3

FIG. 6 illustrates a schematic perspective view of a third embodiment of the nanoelectrode assembly of the invention which is a substantially cylindrical laminate structure. The nanoelectrode assembly 60 comprises a conducting layer 63 deposited on an insulating capping layer 62 which itself is on a solid cylindrical support 61. An insulating capping layer 64 is deposited over the extent of the conducting layer 63 and incorporates an electrical contact 65 for direct and simple connection to an electrochemical measuring device (eg potentiostat). An array of square voids 67 is etched through insulating capping layer 64 and conducting layer 63 and partly through insulating capping layer 62 to an etch depth which is short of the solid cylindrical support 61. The array of square voids 67 extends over only a lower portion 80 of the cylindrical laminate structure. The lower portion 80 is selectively implantable into the body to leave exposed an upper portion 81.

1. A nanoelectrode assembly having a laminate structure comprising:
a first insulating capping layer;
a first conducting layer capped by the first insulating capping layer and substantially sandwiched or encapsulated by at least the first insulating capping layer such as to leave exposed only an electrical contact surface; and
an array of etched voids extending through at least the first insulating capping layer and the first conducting layer, wherein each void is partly bound by a surface of the first conducting layer which acts as an internal submicron electrode upon or adjacent to which is immobilised a glucose sensitive enzyme, wherein in use a glucose-containing bodily fluid passes into the etched voids for exposure to the immobilised glucose sensitive enzyme.

2. The nanoelectrode assembly as claimed in claim 1, wherein in use, relative mass transfer of glucose and oxygen from the bodily fluid to the immobilised glucose sensitive enzyme is unselective.

3. The nanoelectrode assembly as claimed in claim 1, wherein in use, relative mass transfer of glucose and oxygen from the bodily fluid to the immobilised glucose sensitive enzyme is interventionless.

4. The nanoelectrode assembly as claimed in claim 1, wherein in use, relative mass transfer of glucose and oxygen from the bodily fluid to the immobilised glucose sensitive enzyme is unimpeded.

5. The nanoelectrode assembly as claimed in claim 1, which is free of a glucose-restricting membrane.

6. The nanoelectrode assembly as claimed in claim 1, comprising: a plurality of conducting layers (which may be the same or different) including the first conducting layer and a plurality of insulating capping layers (which may be the same or different) including the first insulating capping layer, wherein the plurality of conducting layers and the plurality of insulating capping layers are alternating in the laminate structure, wherein each conducting layer is sandwiched or encapsulated to leave exposed only an electrical contact surface and the array of etched voids extends through the plurality of insulating capping layers and the plurality of conducting layers, wherein each void is partly bound by a surface of each of the plurality of conducting layers which acts as an internal submicron electrode upon or adjacent to which is immobilised the glucose sensitive enzyme.

7. The nanoelectrode assembly as claimed in claim 1, wherein the thickness (wₙ) of the (or each) conducting layer (which may be the same or different) is in the range 0.10 to 75 nm.

8. The nanoelectrode assembly as claimed in claim 1, further comprising:
an insulating substrate layer;
a second insulating capping layer fabricated on the insulating substrate layer, wherein the first conducting layer is fabricated on the second insulating capping layer and is substantially sandwiched or encapsulated by the first insulating capping layer and the second insulating capping layer such as to leave exposed only an electrical contact surface of the first conducting layer.

9. The nanoelectrode assembly as claimed in claim 1, which is substantially planar.

10. The nanoelectrode assembly as claimed in claim 1, which is cylindrical.

11. The nanoelectrode assembly as claimed in claim 1, wherein the glucose sensitive enzyme is glucose oxidase.

12. An electrochemical glucose biosensor comprising:
a nanoelectrode assembly as defined in any preceding claim operable as a working electrode; and
a reference electrode and a counter electrode or a combined counter reference electrode.

13. An apparatus for combating diabetes mellitus in a subject comprising:
an electrochemical glucose biosensor as defined in claim 12 for continuously measuring the glucose level in the subject;
a signal generating device for generating an actuating signal in response to the glucose level exceeding a threshold; and
a delivery device for delivering insulin to the subject in response to the actuating signal, wherein in use the electrochemical glucose biosensor, signal generating device and delivery device communicate in a closed loop.
